(19) United States
(12) Patent Application Publication
(10) Pub. No.: US 2022/0265601 A1
(43) Pub. Date: Aug. 25, 2022

(54) Methods of use and formulations of allosteric modulators of the serotonin, dopamine and other receptor systems for medical, recreational, religious, research and other uses.

(71) Applicant: David Alan Heldreth, JR., Bellevue, WA (US)

(72) Inventor: David Alan Heldreth, JR., Bellevue, WA (US)

(73) Assignee: David Alan Heldreth, JR., Bellevue, WA (US)

(21) Appl. No.: 17/667,147

(22) Filed: Feb. 8, 2022

Related U.S. Application Data
(60) Provisional application No. 63/207,183, filed on Feb. 10, 2021.

(51) Int. Cl. A61K 31/352; A61K 31/05; A61K 31/48; A61K 31/4045; A61K 31/675; A61K 31/404; A61K 33/30; A61K 33/06; A61K 31/015; A61K 31/135; A61K 36/68; A61K 31/473; A61K 31/164; A61K 31/165; A61K 31/4741; A61K 31/704; A61K 36/77

(52) U.S. Cl. CPC A61K 31/352; A61K 31/05; A61K 31/48; A61K 31/4045; A61K 31/675; A61K 31/404; A61K 33/30; A61K 33/06; A61K 31/015; A61K 31/135; A61K 36/68; A61K 31/473; A61K 31/164; A61K 31/165; A61K 31/4741; A61K 31/704; A61K 36/77

(57) ABSTRACT

The invention involves the use of formulations of positive, negative, inverse agonist, or neutral allosteric modulators of receptors such as, but not limited to: 5ht1a/b/c/d, 5ht2a/b/c, 5ht3, 5ht4, 5ht5, 5ht6, 5ht7, dopamine receptors (D1, D2, D3, D4, D5), adrenergic receptors (α1A, α1B, α2A, α2B, α2C, β1, β2), serotonin transporter (SERT), DA transporter (DAT), norepinephrine transporter (NET), imidazoline1 receptor (I1), Sigma receptors (σ1, σ2), delta opioid receptor (DOR), kappa opioid receptor (KOR), mu opioid receptor (MOR), muscarinic receptors (M1, M2, M3, M4, M5), histamine receptors (H1, H2), calcium ion channel (Ca+) and N-methyl D-aspartate (NMDA) glutamate receptor; alone or in combination with items such as, but not limited to: cannabinoids, terpenes, flavonoids, minerals, psychedelic and psychoactive compounds such as, but not limited to 5ht2a receptor agonists or other compounds; for medical, recreational, religious, research and other uses.

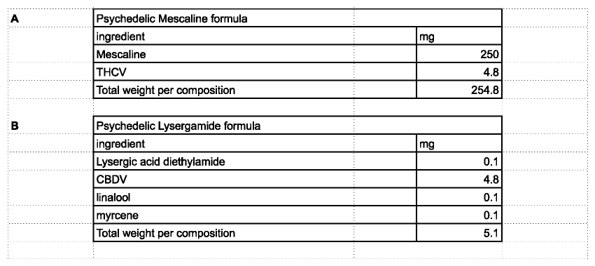

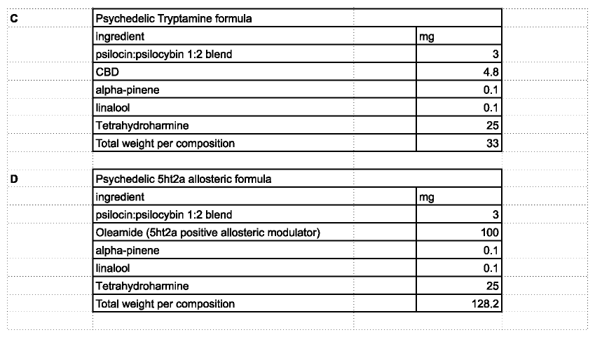

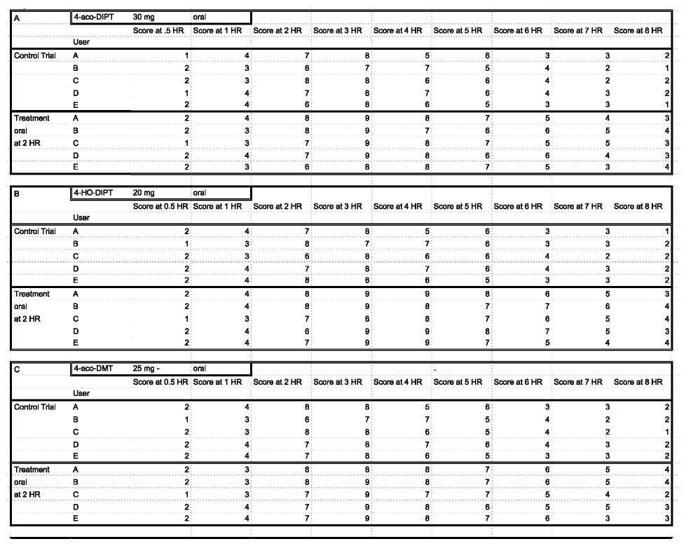

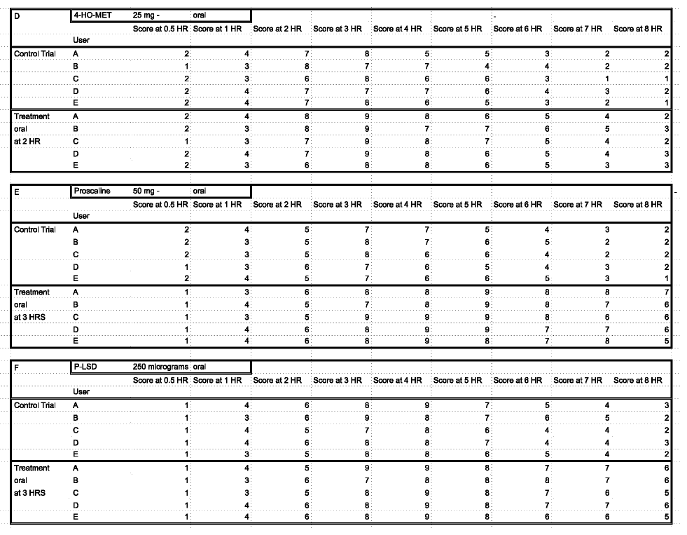

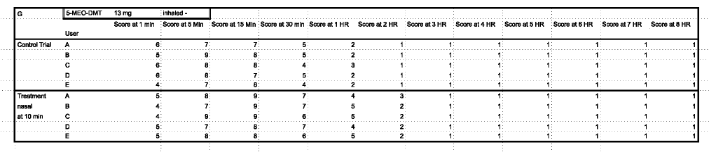

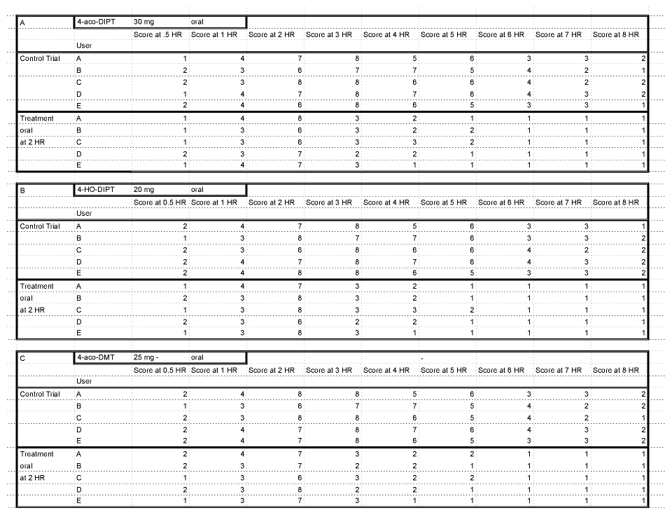

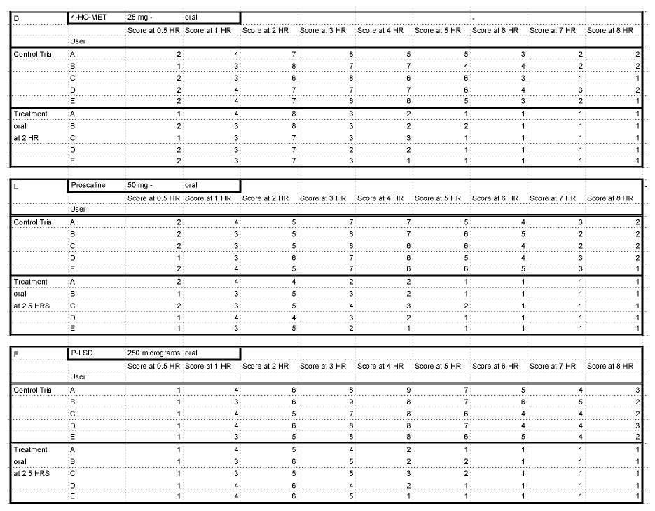

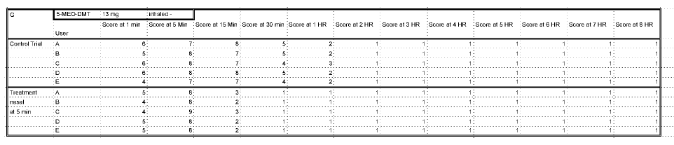

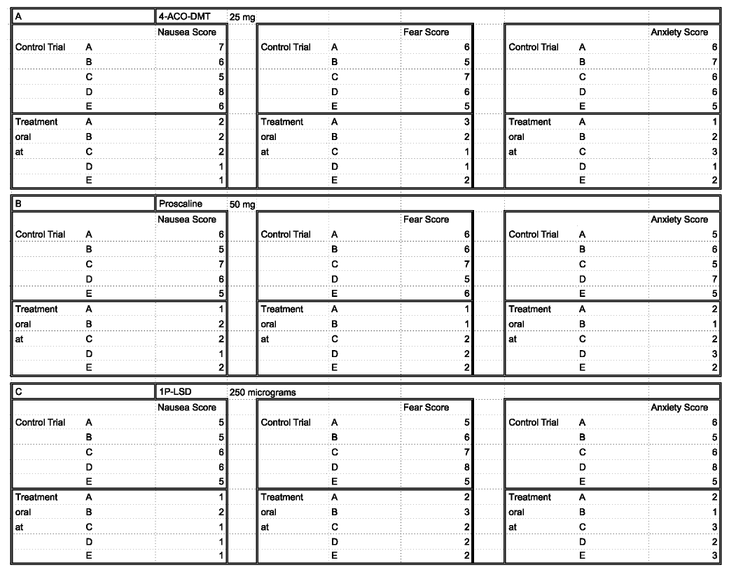

This patent (application) claims prior filing date to provisional patent. Please reference provisional patent title: Psychedelic formulations for medical, recreational, religious, research and other uses. Application Number: 63/207,183.

DESCRIPTION

### FIELD OF THE INVENTION

The invention involves the use of formulations of positive, negative inverse agonist, or neutral allosteric modulators of receptors such as, but not limited to: 5ht1a/b/c/d, 5ht2a/b/c, 5ht3, 5ht4, 5ht5, 5ht6, 5ht7, dopamine receptors (D1, D2, D3, D4, D5), adrenergic receptors (α1A, α1B, α2A, α2B, α2C, β1, β2), serotonin transporter (SERT), DA transporter (DAT), norepinephrine transporter (NET), imidazoline1 receptor (I1), Sigma receptors (σ1, σ2), delta opioid receptor (DOR), kappa opioid receptor (KOR), mu opioid receptor (MOR), muscarinic receptors (M1, M2, M3, M4, M5), histamine receptors (H1, H2), calcium ion channel (Ca+) and N-methyl D-aspartate (NMDA) glutamate receptor; alone or in combination with items such as, but not limited to: cannabinoids, terpenes, flavonoids, minerals, psychedelic and psychoactive compounds such as, but not limited to 5ht2a receptor agonists or other compounds; for medical, recreational, religious, research and other uses.

### BACKGROUND OF THE INVENTION

People across the world are affected by disorders of the mind, such as depression, anxiety, compulsion, and post-traumatic stress disorders and others as listed, outlined and described in the The Diagnostic and Statistical Manual of Mental Disorders. The serotonin system is believed to be involved in many if not all of these conditions.

A variety of compounds are known to modulate activity at the serotonin receptors (5-HT receptor) Pharmaceuticals such as antidepressants, serotonin reuptake inhibitors, selective serotonin reuptake inhibitors, etc. are the current state of art treatments. However, many of these compounds are short acting and come with complicated side effects which make their use and utility limited.

Psilocybin mushrooms or fungi are used recreationally across the United States. Psilocybin is metabolized by the body and liver into psilocin which is the active compound which crosses the blood brain barrier and stimulates the 5-ht receptors. Phenethylamines such as Mescaline normescaline, the 2c series (2C-I/B/E*-NBOH NBOME etc) and MDMA, as well as lysergam ides and ergotamines and beta carbolines have also shown activity on these systems.

The above mentioned compounds provide fast-acting and long-lasting changes to a person's mood, illness etc. These compounds have only minor side effects, low potential for addiction, low potential for abuse, and low risk of toxicity.

These 5-ht receptor compounds have also shown use for treatment of conditions such as migraines, cancer, obesity and have uses as antibiotics, antivirals, antifungals.

The psychedelic compound state of the art focuses primarily on cultivating and consuming mushrooms, cacti or plants. Unfortunately, collecting and ingesting fungi and plants can be dangerous because of difficulties identifying the desired species from similar appearing species. Mistaken identification of mushrooms or plants has led to cases of serious illness and death every year.

Natural products also contain wide variability in composition due to genetics as well as environmental conditions.

As such creation of isolated or near pure compounds and formulation into specific blends would create new opportunities for treatments. Further, these compounds are being legalized for spiritual, religious, recreational and mind exploring purposes in addition to the aforementioned medical uses.

Additionally there is variance in the effects these compounds have based on human genetics and related conditions. Due to this having the ability to reduce the strength of the activity of an agonist on the 5ht2a receptor is important to act as a safety net when giving 5ht2a agonist medications and/or to treat symptoms of diseases interacting with the 5ht2a system in a way that allows fine tuning of the binding. Also having the ability to increase the strength of the activity and binding of an agonist on the 5ht2a receptor is useful to be able to reduce the dose needed of psychedelic compounds which act on the 5ht2a receptor as agonists. This will allow cheaper formulations and reductions in off target side effects. Allosteric binding occurs at a secondary binding site to the orthosteric or main agonist site and thus provides a way for additional compounds to be used in combination to provide ideal binding of receptors for individualized medicine and proper dosing. Allosteric binding affects the affinity and binding rate or activity of the receptor in a variety of ways including, but not limited to changing the shape of the receptor.

These mechanisms of allosteric modulation and activity or biding of receptors by psychedelic and psychoactive compounds apply beyond only the 5ht2a system into the other serotonin receptors as well as several others. It appears that there is an entourage or synergy of activity of compounds on a mixture of receptors in the human body. Beyond the serotonin 2A or 5ht2a receptor other 5ht receptors such as 5ht1, 3, 4, 5, 6, 7 also add unique properties to the subjective experience and therapeutic values. Additionally dopamine receptors (D1, D2, D3, D4, D5), adrenergic receptors (α1A, α1B, α2A, α2B, α2C, β1, β2), serotonin transporter (SERT), DA transporter (DAT), norepinephrine transporter (NET), imidazoline1 receptor (I1), Sigma receptors (σ1, σ2), delta opioid receptor (DOR), kappa opioid receptor (KOR), mu opioid receptor (MOR), muscarinic receptors (M1, M2, M3, M4, M5), histamine receptors (H1, H2), calcium ion channel (Ca+) and the N-methyl D-aspartate (NMDA) glutamate receptor have been shown to be involved in the signaling in the human body during psychedelic compound use.

### SUMMARY OF THE INVENTION

The invention involves the use of formulations of positive, negative or neutral allosteric modulators of receptors such as, but not limited to: 5ht1a/b/c/d, 5ht2b/c, 5ht3, 5ht4, 5ht5, 5ht6, 5ht7, dopamine receptors (D1, D2, D3, D4, D5), adrenergic receptors (α1A, α1B, α2A, α2B, α2C, β1, β2), serotonin transporter (SERT), DA transporter (DAT), norepinephrine transporter (NET), imidazoline1 receptor (I1), Sigma receptors (σ1, σ2), delta opioid receptor (DOR), kappa opioid receptor (KOR), mu opioid receptor (MOR), muscarinic receptors (M1, M2, M3, M4, M5), histamine receptors (H1, H2), calcium ion channel (Ca+) and N-methyl D-aspartate (NMDA) glutamate receptor; alone or in combination with items such as, but not limited to: cannabinoids, terpenes, flavonoids, minerals, psychedelic and psychoactive compounds such as, but not limited to 5ht2a receptor agonists or other compounds; for medical, recreational, religious, research and other uses.

### BRIEF DESCRIPTION OF THE DRAWINGS:

PAGES 1 and 2 drawings show potential formulations of the psychedelic compositions. PAGE 1 SHOWS Diagrams A and B. Diagram A shows a recipe for a mescaline and THCV composition to be taken orally, but may be used in other ways. Diagram B shows a recipe for LSD, CBDV, myrcene and linalool composition to be taken orally, but may be used in other ways. PAGE 2 SHOWS Diagrams C and D. Diagram C shows a recipe for psilocybin, psilocin, CBD, linalool, alpha-pinene and Tetrahydroharmine, composition to be taken orally, but may be used in other ways. Diagram D shows a recipe for shows a recipe for psilocybin, psilocin, CBD, linalool, alpha-pinene and Tetrahydroharmine additionally containing 5ht2a positive allosteric modulator oleamide, composition to be taken orally, but may be used in other ways. Page 3 shows the results of a human experiment on the use of psychedelics 1P-LSD, proscaline and 4-aco-dmt alone or with allosteric modulators to modulate fear, anxiety and nausea which was conducted in February of 2021. The compounds used in addition to the psychedelics were: for Nausea: 5ht3 allosteric modulators: linalool, citral and ginger for nausea; for anxiety and fear: 5ht7 modulator zinc and 5ht1 modulator cannabidiol. Diagram A shows 4-aco-dmt alone or with the above described modulators. Diagram B shows proscaline alone or with the above described modulators. Diagram C shows 1P-LSD alone or with the above described modulators.

Pages 3,4 and 5 shows the results of a human experiment on the use of psychedelics: (diagrams)(A) DIPT, (B)4-HO-DIPT, (C)4-HO-MET, (D)4-ACO-DMT, (E)1P-LSD, (F)proscaline and (G)5-MEO-dmt alone or with positive allosteric modulators.

Pages 6,7 and 8 shows the results of a human experiment on the use of psychedelics: (diagrams)(A) DIPT, (B)4-HO-DIPT, (C)4-HO-MET, (D)4-ACO-DMT, (E)1P-LSD, (F)proscaline and (G)5-MEO-dmt alone or with negative allosteric modulators.

Page 9 shows the results of a human experiment on the use of psychedelics (C) 1P-LSD, (B)proscaline and (A)4-aco-dmt alone or with 5ht3 and 5ht7 allosteric modulators to modulate fear, anxiety and nausea.

### DRAWING SUMMARY

PAGES 1 and 2 drawings show potential formulations of the psychedelic compositions. PAGE 1 SHOWS Diagrams A and B. Diagram A shows a recipe for a mescaline and THCV composition to be taken orally, but may be used in other ways. Diagram B shows a recipe for LSD, CBDV, myrcene and linalool composition to be taken orally, but may be used in other ways. PAGE 2 SHOWS Diagrams C and D. Diagram C shows a recipe for psilocybin, psilocin, CBD, linalool, alpha-pinene and Tetrahydroharmine, composition to be taken orally, but may be used in other ways. Diagram D shows a recipe for shows a recipe for psilocybin, psilocin, CBD, linalool, alpha-pinene and Tetrahydroharmine additionally containing 5ht2a positive allosteric modulator oleamide, composition to be taken orally, but may be used in other ways.

(+)

Pages 3,4 and 5 shows the results of a human experiment on the use of psychedelics: (diagrams)(A) DIPT, (B)4-HO-DIPT, (C)4-HO-MET, (D)4-ACO-DMT, (E)1P-LSD, (F)proscaline and (G)5-MEO-dmt alone or with allosteric modulators on the score of subjective intensity of the experience, hallucinations. The test was conducted in February of 2021 conducted in the US and Canada. The 5ht2a positive allosteric modulator formulation used was: oleamide and monoolein. The formulation was able to repeatedly increase subjective intensity scores and/or extend duration of effect. This can help allow for lower doses to be used which can save cost and/or also reduce off target side effects.

(−)

Pages 6,7 and 8 shows the results of a human experiment on the use of psychedelics: (diagrams)(A) DIPT, (B)4-HO-DIPT, (C)4-HO-MET, (D)4-ACO-DMT, (E)1P-LSD, (F)proscaline and (G)5-MEO-dmt alone or with allosteric modulators on the score of subjective intensity of the experience, hallucinations. The test was conducted in February of 2021 conducted in the US and Canada. The 5ht2a negative allosteric modulator formulation used was: zinc with limonene and cannabidiol. The formulation was able to repeatedly reduce subjective intensity scores.

(=)

Page 9 shows the results of a human experiment on the use of psychedelics 1P-LSD, proscaline and 4-aco-dmt alone or with allosteric modulators to modulate fear, anxiety and nausea which was conducted in February of 2021 conducted in the US and Canada. The compounds used in addition to the psychedelics were: for Nausea: 5ht3 allosteric modulators: linalool, citral and ginger for nausea; for anxiety and fear: 5ht7 modulator zinc and 5ht1a modulator cannabidiol.

Diagram A shows 4-aco-dmt alone or with the above described modulators. Diagram B shows proscaline alone or with the above described modulators. Diagram C shows 1P-LSD alone or with the above described modulators. The modulators were shown to reduce the levels of nausea, fear and anxiety in the experimenters.

### DETAILED DESCRIPTION OF THE INVENTION

The invention involves the use of formulations of 5ht2a receptor allosteric modulators in combination with items such as, but not limited to cannabinoids, terpenes, flavonoids, minerals, psychedelic and psychoactive compounds such as, but not limited to 5ht2a receptor agonists or other compounds; for medical, recreational, religious, research and other uses.

Allosteric modulation is the manipulation of a receptor at a site other than than normal binding site known as the orthosteric site. This is a less utilized method due to only recent discovery of its mechanisms as well as the need to identify these for each receptor. The use of allosteric modulators allows for precise alterations to the activity at the receptor and thus fine tuning of medical effects. Some embodiments utilize compounds which work as positive or negative allosteric modulators of the 5ht2a receptor or other 5ht systems. Allosteric modulation of 5ht2a also includes allosteric modulation through interaction with other receptor systems such as with hetomers or other such items.

In some embodiments the composition will include purified compounds which are either isolated or just purified. In other embodiments raw extracts or ground/processed biomass may be used. Some embodiments include excipients such as water, cyclodextrin, ethanol or other items off of the Food and Drug Administration authorized and approved excipient list. Some embodiments will include utilizing nano technology, encapsulation, beta glucan particles, chitosan, yeast extract, surfactants, binders and other compounds to increase efficiency, availability, release lifespan, release speed among other parameters. In some embodiments compositions are used in foods, drinks, supplements, inhalation products such as, but not limited to: vaporizers, cigarettes, cigars, infused smoking papers, infused smoking blends, cigarillos, vape fluids, eye drops, nasal spray, mouth spray, rectal inhalers or other uses. Some embodiments are for human use and some are for animal uses.

Some embodiments are used to develop vaccines or treatments for virus, fungi or bacteria illness such as, but not limited to: Coronavirus (such as SARS, COVID-19, MERS), HIV, herpes, syphilis,flu virus, swine flu, avian flu, other virus, bacteria or fungi: antibodies-cytokins-proteins-amino acids-DNA-RNA - DNA/RNA to produce antibodies/proteins/amino acids/RNA/DNA; such as for the spike or other proteins used in vaccines or treatment of virus, fungi or bacteria illness. Potential psychedelic and psychoactive compounds can be found below.

COMPOUND LIST

CATHIONE, Psilocybin (4-PO-HO-DMT), psilocin (4-HO-DMT), norpsilocin (ω-N-Methyl-4-hydroxytryptamine), baeocystin (4-PO-DMT), norbaeocystin (4-MeO-MIPT), Aeruginascin (N, N, N-trimethyl-4-phosphoryloxytryptamine), bufotenin (5-HO-DMT), 5-MEO-DMT, N,N-Dimethyltryptamine (N,N-DMT), 4-ACO-DMT, N-acetyl-4-hydroxytryptamine, 4-acetoxy-N-ethyl-N-methyltryptammonium (4-AcO-MET), 4-acetoxy-N,N-diallyltryptammonium (4-AcO-DALT) acid, 4-acetoxy-N-allyl-N-methyltryptammonium (4-AcO-MALT), N,N-dimethyl-N-n-propyltryptammonium (DMPT), N-allyl-N,N-dimethyltryptammonium (DMALT), 4-HO-TMT (4-hydroxy-N,N,N-trimethyltryptamine), N-methyltryptamine, 4-HO-NMT, 5-HO-NMT, 5-PO-HO-DMT, 4-PO-HO-NMT, 5-PO-HO-NMT, 4-HO-NMT, 4-HO-DMT, N-Methyl-4-phosphoryloxytryptamine, N,N-diethyl-tryptamine, 4-hydroxy-N,N-diethyltryptamine, 4-phosphoryloxy-N,N-diethyltryptamine, 5-HO-DET, 5-PO-DET, 4-PO-HO-DET, 5-PO-HO-DET, 5-PO-HO-DMT, 5-ACO-DMT, 4-ACO-NMT, 4-ACO-DET, 5-ACO-DET, 5-ACO-NMT, 4-hydroxy-6-methyl-L-tryptophan, 6-methyl psilocybin, 6-methyl psilocin, 6-methyl norpsilocin, 6-methyl baeocystin, 6-methyl norbaeocystin, 6-methyl Aeruginascin, 6-methyl bufotenin, 6-methylindole, 3-methylindole, 3-methyl baeocystin, 3-methyl norbaeocystin, 3-methyl Aeruginascin, 3-methyl bufotenin, 7-methylindole, 7-methyl baeocystin, 7-methyl norbaeocystin, 7-methyl Aeruginascin, 7-methyl bufotenin, 4-hydroxy-3-methyl-L-tryptophan, 4-hydroxy-7-methyl-L-tryptophan, 5-ht, gaba (gamma-Aminobutyric acid), serotonin, dopamine, epinephrine, oxytocin, tryptamine, ergotamines/lysergides such as, but not limited to LSD, LDZ,1P-LSD, LSA, ergotaline. 5-Bromo-DMT, 5,6-Dibromo-DMT, 4-aco-mpt, 4-aco-mipt, 4-aco-ept, 4-aco-dipt, 4-aco-dpt, Phenethylamines including, but not limited to: mescaline, normescaline, 2c-i,2c-b, 2c-e, Beta carbolines and maoi inhibitors including, but not limited to: harmaline, tetraharmaline, norharmane, perlolyrine, tetrahydroharmine, harmane, harmine, harmol.

Compounds found to be Monoamine oxidase inhibitors. Compounds found to be active at the 5ht2a, 5ht2b, 5ht2c receptors.

Kratom compounds such as, but not limited to: mitragynine, mitraphylline, 7-hydroxymitragynine, raubasine and corynantheidine.

Salvia divinorum compounds inducing, but not limited to diterpenoids such as salvinorin A and other isomers or analogs.

Cocaine and related analogs including other tropanes.

Iboga alkaloids such as, but not limited to: Coronaridine, Ibogamine, Voacangine, Ibogaine, conopharyngine, ibogaline, stemmadenine.

Cannabinoids including, but not limited to: endocannabinoids, phytocannabinoids, compounds which are ligands at CB1 or CB2 receptors such as, but not limited to: 2-AG, anandamide, CBD cannabidiol, THC (Tetrahydrocannabinol), Tetrahydrocannabivarin THCV, CBDV Cannabidivarin , tetrahydrocannabiphorol THCP, cannabidiphorol CBDP, hexyl CBD, THC acetates, CBD acetates, Cannabigerovarin CBGV, CBG cannabigerol, CBG acetates, heli-CBG, (CBC) cannabichromene, Cannabichromevarin CBCV, cannabinol CBN, beta caryophyllene, also butyl, hexyl, hepyl, octyl, deca, versions. Cannabinoids include all isomers including delta-8, delta-9, delta-10 and beyond such as for THC, but also including other cannabinoids.

Terpenes and terpenoids, such as, but not limited to: limonene, alpha-pinene, myrcene, linalool, terpinolene and all isomers of such compounds.

Antibiotics or antifungals such as penicillin, azithromycin or miconazole.

Coronavirus (such as SARS, COVID-19, MERS), HIV, herpes, syphilis,flu virus, swine flu, avian flu, other virus, bacteria or fungi: antibodies-proteins-amino acids-DNA-RNA - DNA/RNA to produce antibodies/proteins/amino acids/RNA/DNA; such as for the spike or other proteins used in vaccines or treatment of virus, fungi or bacteria illness.

AL-LAD, DBT, DET, DiPT, 5-MeO-α-MT, DMT, 2,α-DMT, α,N-DMT, DPT, EiPT, α-ET, ETH-LAD, Harmaline, Harmine, 4-HO-DBT, 4-HO-DET, 4-HO-DiPT, 4-HO-DMT, 5-HO-DMT, 4-HO-DPT, 4-HO-MET, 4-HO-MiPT, 4-HO-MPT, 4-HO-pyr-T, Ibogaine, LSD, MBT, 4,5-MDO-DiPT, 5,6-MDO-DiPT, 4,5-MDO-DMT, 5,6-MDO-DMT, 5,6-MDO-MiPT, 2-Me-DET, 2-Me-DMT, Melatonin, 5-MeO-DET, 5-MeO-DiPT, 5-MeO-DMT, 4-MeO-MiPT, 5-MeO-MiPT, 5,6-MeO-MiPT, 5-MeO-NMT, 5-MeO-pyr-T, 6-MeO-THH, 5-MeO-TMT, 5-MeS-DMT, MiPT, α-MT, NET, NMT, PRO-LAD, pyr-T, Tryptamine, Tetrahydroharmine, α,N,O-TMS, α,N,N-TMT⋅2,N,N-TMT⋅5,N,N-TMT⋅4-Acetoxy-DMT⋅4-Acetoxy-DET⋅4-Acetoxy-DIPT⋅4-HO-5-MeO-DMT⋅α-ET⋅α-MT⋅Baeocystin⋅Bufotenin⋅DBT⋅DET⋅DIPT⋅DMT⋅EiPT⋅PiPT⋅Ethocin⋅Ethocybin⋅Iprocin⋅4-HO-MET⋅4-HO-MiPT⋅MET⋅MIPT⋅5-Me-MIPT⋅5-MeO-α-ET⋅5-MeO-α-MT⋅5-MeO-DALT⋅5-MeO-DET⋅5-MeO-DIPT⋅5-MeO-DMT⋅5-MeO-DPT⋅5MeO-DPT⋅5-MeO-MET⋅5-MeO-MIPT⋅5-MeO-αN,N-TMT⋅5-MeO-2,N,N-TMT⋅Miprocin⋅Norbaeocystin⋅Psilocin⋅Psilocybin,4-HO-MALT,

4-Acetoxy-DET⋅4-Acetoxy-DIPT⋅4-Acetoxy-DMT⋅4-HO-DIPT⋅5-Bromo-DMT⋅5-Fluora-α-MT⋅5-MeO-α-ET⋅5-MeO-α-MT⋅5-MeO-DALT⋅5-MeO-DET⋅5-MeO-DIPT⋅5MeO-DMT⋅5-MeO-DPT⋅5-MeO-MIPT⋅α-ET⋅αMT⋅Baeocystin⋅Bufotenin⋅DET⋅DiPT⋅DMT⋅DPT⋅Ethocybin⋅EiPT⋅Ethocin⋅Ibogaine⋅Iprocin⋅MET⋅MiPT⋅Miprocin⋅Melatonin⋅NMT Norbaeocystin⋅Normelatonin⋅PiPT⋅Psilocin⋅Psilocybin⋅Rizatriptan⋅Serotonin⋅Sumatriptan⋅Tryptamine⋅

| | | |
|---|---|---|
| 1 | AEM | alpha-Ethyl-3,4,5-trimethoxy-PEA |
| 2 | AL | 4-Allyloxy-3,5-dimethoxy-PEA |
| 3 | ALEPH | 4-Methylthio-2,5-dimethoxy-A |
| 4 | ALEPH-2 | 4-Ethylthio-2,5-dimethoxy-A |
| 5 | ALEPH-4 | 4-Isopropylthio-2,5-dimethoxy-A |
| 6 | ALEPH-6 | 4-Phenylthio-2,5-dimethoxy-A |
| 7 | ALEPH-7 | 4-Propylthio-2,5-dimethoxy-A |
| 8 | ARIADNE | 2,5-Dimethoxy-alpha-ethyl-4-methyl-PEA |
| 9 | ASB | 3,4-Diethoxy-5-methoxy-PEA |
| 10 | B | 4-Butoxy-3,5-dimethoxy-PEA |
| 11 | BEATRICE | 2,5-Dimethoxy-4,N-dimethyl-A |
| 12 | BIS-TOM | 2,5-Bismethylthio-4-methyl-A |
| 13 | BOB | 4-Bromo-2,5, beta-trimethoxy-PEA |
| 14 | BOD | 2,5,beta-Trimethoxy-4-methyl-PEA |
| 15 | BOH | beta-Methoxy-3,4-methylenedioxy-PEA |
| 16 | BOHD | 2,5-Dimethoxy-beta-hydroxy-4-methyl-PEA |
| 17 | BOM | 3,4,5, beta-Tetram ethoxy-P E A |
| 18 | 4-Br-3,5-DMA | 4-Bromo-3,5-dimethoxy-A |
| 19 | 2-Br-4,5-MDA | 2-Bromo-4,5-methylenedioxy-A |
| 20 | 2C-B | 4-Bromo-2,5-dimethoxy-PEA |
| 21 | 3C-BZ | 4-Benzyloxy-3,5-dimethoxy-A |
| 22 | 2C-C | 4-Chloro-2,5-dimethoxy-PEA |
| 23 | 2C-D | 4-Methyl-2,5-dimethoxy-PEA |
| 24 | 2C-E | 4-Ethyl-2,5-dimethoxy-PEA |
| 25 | 3C-E | 4-Ethoxy-3,5-dimethoxy-A |
| 26 | 2C-F | 4-Fluoro-2,5-dimethoxy-PEA |
| 27 | 2C-G | 3,4-Dimethyl-2,5-dimethoxy-PEA |
| 28 | 2C-G-3 | 3,4-Trimethylene-2,5-dimethoxy-PEA |
| 29 | 2C-G-4 | 3,4-Tetramethylene-2,5-dimethoxy-PEA |
| 30 | 2C-G-5 | 3,4-Norbornyl-2,5-dimethoxy-PEA |
| 31 | 2C-G-N | 1,4-Dimethoxynaphthyl-2-ethylamine |
| 32 | 2C-H | 2,5-Dimethoxy-PEA |
| 33 | 2C-I | 4-lodo-2,5-dimethoxy-PEA |
| 34 | 2C-N | 4-Nitro-2,5-dimethoxy-PEA |
| 35 | 2C-O-4 | 4-Isopropoxy-2,5-dimethoxy-PEA |
| 36 | 2C-P | 4-Propyl-2,5-dimethoxy-PEA |
| 37 | CPM | 4-Cyclopropylmethoxy-3,5-dimethoxy-PEA |
| 38 | 2C-SE | 4-Methylseleno-2,5-dimethoxy-PEA |
| 39 | 2C-T | 4-Methylthio-2,5-dimethoxy-PEA |
| 40 | 2C-T-2 | 4-Ethylthio-2,5-dimethoxy-PEA |
| 41 | 2C-T-4 | 4-Isopropylthio-2,5-dimethoxy-PEA |
| 42 | psi-2C-T-4 | 4-Isopropylthio-2,6-dimethoxy-PEA |
| 43 | 2C-T-7 | 4-Propylthio-2,5-dimethoxy-PEA |
| 44 | 2C-T-8 | 4-Cyclopropylmethylthio-2,5-dimethoxy-PEA |
| 45 | 2C-T-9 | 4-(t)-Butylthio-2,5-dimethoxy-PEA |
| 46 | 2C-T-13 | 4-(2-Methoxyethylthio)-2,5-dimethoxy-PEA |
| 47 | 2C-T-15 | 4-Cyclopropylthio-2,5-dimethoxy-PEA |
| 48 | 2C-T-17 | 4-(s)-Butylthio-2,5-dimethoxy-PEA |
| 49 | 2C-T-21 | 4-(2-Fluoroethylthio)-2,5-dimethoxy-PEA |
| 50 | 4-D | 4-Trideuteromethyl-3,5-dimethoxy-PEA |
| 51 | beta-D | beta,beta-Dideutero-3,4,5-trimethoxy-PEA |
| 52 | DESOXY | 4-Methyl-3,5-Dimethoxy-PEA |
| 53 | 2,4-DMA | 2,4-Dimethoxy-A |
| 54 | 2,5-DMA | 2,5-Dimethoxy-A |
| 55 | 3,4-DMA | 3,4-Dimethoxy-A |
| 56 | DMCPA | 2-(2,5-Dimethoxy-4-methylphenyl)- |
| | | cyclopropylamine |
| 57 | DME | 3,4-Dimethoxy-beta-hydroxy-PEA |
| 58 | DMMDA | 2,5-Dimethoxy-3,4-methylenedioxy-A |
| 59 | DMMDA-2 | 2,3-Dimethoxy-4,5-methylenedioxy-A |
| 60 | DMPEA | 3,4-Dimethoxy-PEA |
| 61 | DOAM | 4-Amyl-2,5-dimethoxy-A |
| 62 | DOB | 4-Bromo-2,5-dimethoxy-A |
| 63 | DOBU | 4-Butyl-2,5-dimethoxy-A |
| 64 | DOC | 4-Chloro-2,5-dimethoxy-A |
| 65 | DOEF | 4-(2-Fluoroethyl)-2,5-dimethoxy-A |
| 66 | DOET | 4-Ethyl-2,5-dimethoxy-A |
| 67 | DOI | 4-lodo-2,5-dimethoxy-A |
| 68 | DOM (STP) | 4-Methyl -2,5-dimethoxy-A |
| 69 | psi-DOM | 4-Methyl -2,6-dimethoxy-A |
| 70 | DON | 4-Nitro-2,5-dimethoxy-A |
| 71 | DOPR | 4-Propyl-2,5-dimethoxy-A |
| 72 | E | 4-Ethoxy-3,5-dimethoxy-PEA |
| 73 | EEE | 2,4,5-Triethoxy-A |
| 74 | EEM | 2,4-Diethoxy-5-methoxy-A |
| 75 | EME | 2,5-Diethoxy-4-methoxy-A |
| 76 | EMM | 2-Ethoxy-4,5-dimethoxy-A |
| 77 | ETHYL-J | N, alpha-diethyl-3,4-methylenedioxy-PEA |
| 78 | ETHYL-K | N-Ethyl-alpha-propyl-3,4-methylenedioxy-PEA |
| 79 | F-2 | Benzofuran-2-methyl-5-methoxy- |
| | | 6-(2-aminopropane ) |
| 80 | F-22 | Benzofuran-2,2-dimethyl-5- |
| | | methoxy-6-(2-aminoprop ane) |
| 81 | FLEA | N-Hydroxy-N-methyl-3,4-methylenedioxy-A |
| 82 | G-3 | 3,4-Trimethylene-2,5-dimethoxy-A |
| 83 | G-4 | 3,4-Tetramethylene-2,5-dimethoxy-A |
| 84 | G-5 | 3,4-Norbornyl-2,5-dimethoxy-A |
| 85 | GANESHA | 3,4-Dimethyl-2,5-dimethoxy-A |
| 86 | G-N | 1,4-Dimethoxynaphthyl-2-isopropylamine |
| 87 | HOT-2 | 2,5-Dimethoxy-N-hydroxy-4-ethylthio-PEA |
| 88 | HOT-7 | 2,5-Dimethoxy-N-hydroxy-4-(n)-propylthio-PEA |
| 89 | HOT-17 | 2,5-Dimethoxy-N-hydroxy-4-(s)-butylthio-PEA |
| 90 | IDNNA | 2,5-Dimethoxy-N,N-dimethyl-4-iodo-A |
| 91 | IM | 2,3,4-Trimethoxy-PEA |
| 92 | IP | 3,5-Dimethoxy-4-isopropoxy-PEA |
| 93 | IRIS | 5-Ethoxy-2-methoxy-4-methyl-A |
| 94 | J | alpha-Ethyl-3,4-methylenedioxy-PEA |
| 95 | LOPHOPHINE | 3-Methoxy-4,5-methylenedioxy-PEA |
| 96 | M | 3,4,5-Trimethoxy-PEA |
| 97 | 4-MA | 4-Methoxy-A |
| 98 | MADAM-6 | 2,N-Dimethyl-4,5-methylenedioxy-A |
| 99 | MAL | 3,5-Dimethoxy-4-methallyloxy-PEA |
| 100 | MDA | 3,4-Methylenedioxy-A |
| 101 | MDAL | N-Allyl-3,4-methylenedioxy-A |
| 102 | MDBU | N-Butyl-3,4-methylenedioxy-A |
| 103 | MDBZ | N-Benzyl-3,4-methylenedioxy-A |
| 104 | MDCPM | N-Cyclopropylmethyl-3,4-methylenedioxy-A |
| 105 | MDDM | N,N-Dimethyl-3,4-methylenedioxy-A |
| 106 | MDE | N-Ethyl-3,4-methylenedioxy-A |
| 107 | MDHOET | N-(2-Hydroxyethyl)-3,4-methylenedioxy-A |
| 108 | MDIP | N-Isopropyl-3,4-methylenedioxy-A |
| 109 | MDMA | N-Methyl-3,4-methylenedioxy-A |
| 110 | MDMC | N-Methyl-3,4-ethylenedioxy-A |
| 111 | MDMEO | N-Methoxy-3,4-methylenedioxy-A |
| 112 | MDMEOET | N-(2-Methoxyethyl)-3,4-methylenedioxy-A |
| 113 | MDMP | alpha,alpha, N-Trimethyl-3,4- |
| | | methylenedioxy-PEA |
| 114 | MDOH | N-Hydroxy-3,4-methylenedioxy-A |
| 115 | MDPEA | 3,4-Methylenedioxy-PEA |
| 116 | MDPH | alpha,alpha-Dimethyl-3,4-methylenedioxy-PEA |
| 117 | MDPL | N-Propargyl-3,4-methylenedioxy-A |
| 118 | MDPR | N-Propyl-3,4-methylenedioxy-A |
| 119 | ME | 3,4-Dimethoxy-5-ethoxy-PEA |
| 120 | MEDA | 3-methoxy-4,5-Ethylenedioxy-A |
| | | [Erowid corrected] |
| 121 | MEE | 2-Methoxy-4,5-diethoxy-A |
| 122 | MEM | 2,5-Dimethoxy-4-ethoxy-A |
| 123 | MEPEA | 3-Methoxy-4-ethoxy-PEA |
| 124 | META-DOB | 5-Bromo-2,4-dimethoxy-A |
| 125 | META-DOT | 5-Methylthio-2,4-dimethoxy-A |
| 126 | METHYL-DMA | N-Methyl-2,5-dimethoxy-A |
| 127 | METHYL-DOB | 4-Bromo-2,5-dimethoxy-N-methyl-A |
| 128 | METHYL-J | N-Methyl-alpha-ethyl-3,4-methylenedioxy-PEA |
| 129 | METHYL-K | N-Methyl-alpha-propyl-3,4-methylenedioxy-PEA |
| 130 | METHYL-MA | N-Methyl-4-methoxy-A |
| 131 | METHYL- | N-Methyl-2-methoxy-4,5-methylenedioxy-A |
| | MMDA-2 | |
| 132 | MMDA | 3-Methoxy-4,5-methylenedioxy-A |
| 133 | MMDA-2 | 2-Methoxy-4,5-methylenedioxy-A |
| 134 | MMDA-3a | 2-Methoxy-3,4-methylenedioxy-A |
| 135 | MMDA-3b | 4-Methoxy-2,3-methylenedioxy-A |
| 136 | MME | 2,4-Dimethoxy-5-ethoxy-A |
| 137 | MP | 3,4-Dimethoxy-5-propoxy-PEA |
| 138 | MPM | 2,5-Dimethoxy-4-propoxy-A |
| 139 | ORTHO-DOT | 2-Methylthio-4,5-dimethoxy-A |
| 140 | P | 3,5-Dimethoxy-4-propoxy-PEA |
| 141 | PE | 3,5-Dimethoxy-4-phenethyloxy-PEA |
| 142 | PEA | PEA |
| 143 | PROPYNYL | 4-Propynyloxy-3,5-dimethoxy-PEA |
| 144 | SB | 3,5-Diethoxy-4-methoxy-PEA |
| 145 | TA | 2,3,4,5-Tetramethoxy-A |
| 146 | 3-TASB | 4-Ethoxy-3-ethylthio-5-methoxy-PEA |
| 172 | 5-TOM | 2-Methoxy-4-methyl-5-methylthio-A |
| 173 | TOMSO | 2-Methoxy-4-methyl-5-methylsulfinyl-A |
| 174 | TP | 4-Propylthio-3,5-dimethoxy-PEA |
| 175 | TRIS | 3,4,5-Triethoxy-PEA |
| 176 | 3-TSB | 3-Ethoxy-5-ethylthio-4-methoxy-PEA |
| 177 | 4-TSB | 3,5-Diethoxy-4-methylthio-PEA |
| 178 | 3-T-TRIS | 4,5-Diethoxy-3-ethylthio-PEA |
| 179 | 4-T-TRIS | 3,5-Diethoxy-4-ethylthio-PEA |

Includes all: phenethylamines in 2(X) series such as 2-CI including all NBOME, NBOH, and other analogs.

Additionally includes all: (4-acetoxy) (4-hydroxy)(dimethyl) (diethyl) (N-methyl-N-ethyl) (N-methyl)(N-methyl-N-isopropyl)(N,N-diisopropyl) variations of compounds in metabolite list.

Additionally includes all: functional group variants, fumerates, fumerics, idoines, hydrofumarates, deneutered or not, salts, acids, isomers, analogs, precursors, further metabolites of biosynthetic or synthetic pathways. Also: Zinc, Magnesium Sulfate,Carvelidol,lbuprofen,Mitragynine,CorynantheidinePaynantheine,Sarpogrelate, Potassium, Monoolein,Oleamide,(R) glaucine,Ebelactone B,Bacopa monnieri,aescin,Horse chestnut, and others. zinc, oleamide, (R) Glaucine, (s) Glaucine, isolaureline, dicentrine, amides active on cb1 or cb2, 2AG, anandamide, eugenol, capsaicin, Monoolein, citral, linalool, boldine, eucalyptol, citronellol, geraniol, gingerols, thujone, zingerone, shogaols, menthol, cannabigerol, cannabidiol, cannabrimonene, other cannabinoids,oleamide, THC, 2-AG, oleylethanolamide (OEA), and 2-oleoyl glycerol (2OG), Potassium, Monoolein, (R) Glaucine, (s) Glaucine, isolaureline, dicentrine, amides, lipids, fatty acid amides, amides active on cb1 or cb2, 2AG, anandamide, Ebelactone B,Bacopa monnieri compounds, aescin, horse chestnut compounds or others. Diphenhydramine and other antihistamines.

1. A composition comprising:
A) a formulation of allosteric modulator or modulators of receptor or receptors such as, but not limited to:
a) 5ht1a/b/c/d, 5ht2a/b/c, 5ht3, 5ht4, 5ht5, 5ht6, 5ht7,
b) dopamine receptors (D1, D2, D3, D4, D5),
c) adrenergic receptors (α1A, α1B, α2A, α2B, α2C, β1, α2),
d) serotonin transporter (SERT), DA transporter (DAT), norepinephrine transporter (NET),
e) imidazoline1 receptor (I1), Sigma receptors (σ1, σ2),
f) delta opioid receptor (DOR), kappa opioid receptor (KOR), mu opioid receptor (MOR),
g) muscarinic receptors (M1, M2, M3, M4, M5),
h) histamine receptors (H1, H2),
i) calcium ion channel (Ca+) and N-methyl D-aspartate (NMDA) glutamate receptor
Which comprises items such as, but not limited to: zinc, oleamide, (R) Glaucine, (s) Glaucine, isolaureline, dicentrine, amides active on cb1 or cb2, 2AG, anandamide, eugenol, capsaicin, Monoolein, citral, linalool, boldine, eucalyptol, citronellol, geraniol, gingerols, thujone, zingerone, shogaols, menthol, cannabigerol, cannabidiol, cannabrimonene, other cannabinoids, oleamide, THC, 2-AG, oleylethanolamide (OEA), and 2-oleoyl glycerol (2OG), Potassium, Monoolein, (R) Glaucine, (s) Glaucine, isolaureline, dicentrine, amides, lipids, fatty acid amides, amides active on cb1 or cb2, 2AG, anandamide, Ebelactone B,Bacopa monnieri compounds, aescin, horse chestnut compounds or others; AND OPTIONALLY
j) OPTIONALLY further comprising: A terpene/terpenoid or terpenes/terpenoids or a blend thereof
k) OPTIONALLY further comprising: a cannabis extract(s) or cannabinoid or cannabinoids such as, but not limited to CBD, CBDV, CBDP, THC, THCV, THCP, CBG, CBGV, CBGP, CBC, CBCV, CBCP, CBN, CBNV as well as all acidic (non-decarboxylated forms) as well as endocannabinoids, such as anandamide, oleamide, 2AG or others,
l) OPTIONALLY further comprising: Diphenhydramine and other antihistamines;
which is for animal or human consumption wherein the composition(s) are used for one of the following or more reasons:
a) treat(s) or reduces symptoms of or used to develop vaccines or treatments for virus, fungi or bacteria illness such as, but not limited to: Coronavirus (such as SARS, COVID-19, MERS), HIV, herpes, syphilis, flu virus, swine flu, avian flu, other virus, bacteria or fungi: antibodies-cytokins-proteins-amino acids-DNA-RNA - DNA/RNA to produce antibodies/proteins/amino acids/RNA/DNA; such as for the spike or other proteins used in vaccines or treatment of virus, fungi or bacteria illness,
b) treat(s) or reduces symptoms of: cancer, pain, migraine/headaches, brain injuries, nausea or related conditions,
c) Is used for increasing IQ, increasing productivity, religious, spiritual, psychonaut, mind exploring or recreational uses,
d) treat(s) or reduces symptoms of: psychedelic use, 5ht2a agonists use, hallucinations related symptoms,
e) treat(s) or reduces symptoms of conditions listed in the Diagnostic and Statistical Manual of Mental Disorders by the American Psychiatric Association, such as, but notl limited to: depression, anxiety, schizophrenia, PTSD, sexual dysfunction, substance abuse, hallucinations and more.

2. The composition of claim 1 further comprising: A terpene/terpenoid or terpenes/terpenoids or a blend thereof.

3. The composition of claim 1 further comprising: a cannabis extract(s) or cannabinoid or cannabinoids such as, but not limited to CBD, CBDV, CBDP, THC, THCV, THCP, CBG, CBGV, CBGP, CBC, CBCV, CBCP, CBN, CBNV as well as all acidic (non-decarboxylated forms) as well as endocannabinoids, such as anandamide, oleamide, 2AG or others.

4. A method of using the composition of claim 1 comprising the use of the composition after the use of one or more compounds from these classes or lists: tryptamines, lysergamides, phenethylamines, ergotamines, beta carbolines or maois, or other compounds active on the 5ht2a receptor such as, but not limited to those from the COMPOUND LIST in the patent description.

5. The method of claims 1 in which the composition is used in vaporizers, cigarettes, inhalers, eye drops, nasal drops, insufflated, mouth sprays, sublingually, in tinctures, capsules, gel tabs, tablets, topically, intravenously, rectally, orally, in food, supplements, drinks, alcoholic beverages, teas, juices, fermented, distilled or other related methods.

6. The method and composition of claims 1 in which the composition includes additives, surfactants, cyclodextrin, polysorbates, emulsifiers nano technology, encapsulation, beta glucan particles, chitosan, yeast extract, binders and other compounds to increase efficiency, stability, availability, release lifespan, release speed among other parameters.

7. A composition comprising:
a) a 5ht2a receptor a positive allosteric modulator or modulators such as, but not limited to: oleamide, THC, 2-AG, oleylethanolamide (OEA), and 2-oleoyl glycerol (2OG), Potassium, Monoolein, (R) Glaucine, (s) Glaucine, isolaureline, dicentrine, amides, lipids, fatty acid amides, amides active on cb1 or cb2, 2AG, anandamide, Ebelactone B,Bacopa monnieri compounds, aescin, horse chestnut compounds, and others, AND optionally
b) further comprising: A terpene/terpenoid or terpenes/terpenoids or a blend thereof
c) further comprising: a cannabis extract(s) or cannabinoid or cannabinoids such as, but not limited to CBD, CBDV, CBDP, THC, THCV, THCP, CBG, CBGV, CBGP, CBC, CBCV, CBCP, CBN, CBNV as well as all acidic (non-decarboxylated forms) as well as endocannabinoids, such as anandamide, oleamide, 2AG or others.
which is for animal or human consumption wherein the composition(s) are used for one of the following or more reasons:
a) treat(s) or reduces symptoms of or used to develop vaccines or treatments for virus, fungi or bacteria illness such as, but not limited to: Coronavirus (such as SARS, COVID-19, MERS), HIV, herpes, syphilis,flu virus, swine flu, avian flu, other virus, bacteria or fungi: antibodies-cytokins-proteins-amino acids-DNA-RNA - DNA/RNA to produce antibodies/proteins/amino acids/RNA/DNA; such as for the spike or other proteins used in vaccines or treatment of virus, fungi or bacteria illness,
b) treat(s) or reduces symptoms of: cancer, pain, migraine/headaches, brain injuries, nausea or related conditions,
c) Is used for increasing IQ, increasing productivity, religious, spiritual, psychonaut, mind exploring or recreational uses,
d) treat(s) or reduces symptoms of: psychedelic use, 5ht2a agonists use, hallucinations related symptoms,
e) treat(s) or reduces symptoms of conditions listed in the Diagnostic and Statistical Manual of Mental Disorders by the American Psychiatric Association, such as, but notl limited to: depression, anxiety, schizophrenia, PTSD, sexual dysfunction, substance abuse, hallucinations and more.

8. A composition of claim 7 in which the allosteric modulator of the 5ht2a receptor is:
a) not a NMDA receptor agonist
b) a NMDA receptor antagonist.

9. The method of using the composition of claim 7 comprising the use of the composition after or before the use of one or more compounds from these classes or lists: tryptamines, lysergamides, phenethylamines, ergotamines, beta carbolines or maois, or other compounds active on the 5ht2a receptor such as, but not limited to those from the COMPOUND LIST in the patent description.

10. The method and composition of claim 7 in which the composition is used in vaporizers, cigarettes, inhalers, eye drops, nasal drops, insufflated, mouth sprays, sublingually, in tinctures, capsules, gel tabs, tablets, topically, intravenously, rectally, orally, in food, supplements, drinks, alcoholic beverages, teas, juices, fermented, distilled or other related methods.

11. The method and composition of claim 7 in which the composition includes additives, surfactants, cyclodextrin, polysorbates, emulsifiers nano technology, encapsulation, beta glucan particles, chitosan, yeast extract, binders and other compounds to increase efficiency, stability, availability, release lifespan, release speed among other parameters.

12. A composition comprising:
a) a 5ht2a receptor a negative allosteric modulator or modulators such as, but not limited to: zinc, magnesium, carvedilol, cannabidiol, limonene, THCV or other items, AND optionally
b) further comprising: A terpene/terpenoid or terpenes/terpenoids or a blend thereof
c) further comprising: a cannabis extract(s) or cannabinoid or cannabinoids such as, but not limited to CBD, CBDV, CBDP, THC, THCV, THCP, CBG, CBGV, CBGP, CBC, CBCV, CBCP, CBN, CBNV as well as all acidic (non-decarboxylated forms) as well as endocannabinoids, such as anandamide, oleamide, 2AG or others.
which is for animal or human consumption wherein the composition(s) are used for one of the following or more reasons:
a)Is used for increasing IQ, increasing productivity, religious, spiritual, psychonaut, mind exploring or recreational uses,
b) reduces symptoms of: psychedelic use, 5ht2a agonists use, cannabis use, hallucinations or related symptoms.

13. A composition of claim 12 in which the allosteric modulator of the 5ht2a receptor is:
a) not a NMDA receptor agonist
b) a NMDA receptor antagonist.

14. The method of using the composition of claim 12 comprising the use of the composition after or before the use of one or more compounds from these classes or lists: tryptamines, lysergamides, phenethylamines, ergotamines, beta carbolines or maois, or other compounds active on the 5ht2a receptor such as, but not limited to those from the COMPOUND LIST in the patent description.

15. The method and composition of claim 12 in which the composition is used in vaporizers, cigarettes, inhalers, eye drops, nasal drops, insufflated, mouth sprays, sublingually, in tinctures, capsules, gel tabs, tablets, topically, intravenously, rectally, orally, in food, supplements, drinks, alcoholic beverages, teas, juices, fermented, distilled or other related methods.

16. The method and composition of claim 12 in which the composition includes additives, surfactants, cyclodextrin, polysorbates, emulsifiers nano technology, encapsulation, beta glucan particles, chitosan, yeast extract, binders and other compounds to increase efficiency, stability, availability, release lifespan, release speed among other parameters.
